(19) United States
(12) Patent Application Publication
(10) Pub. No.: US 2005/0226911 A1
(43) Pub. Date: Oct. 13, 2005

(54) Article for inhibiting microbial growth in physiological fluids

(76) Inventors: Joseph F. Bringley, Rochester, NY (US); David L. Patton, Webster, NY (US); Richard W. Wien, Pittsford, NY (US); Yannick J. F. Lerat, Mellecey (FR)

Correspondence Address: Pamela R. Crocker;Patent Legal Staff, Eastman Kodak Company, 343 State Street, Rochester, NY, 14650-2201, US

(21) Appl. No.: 10/822,945

(22) Filed: Apr. 13, 2004

(51) Int. Cl. A61K 9/70; A61K 9/14

(52) U.S. Cl. 424/443; 424/489

(57) ABSTRACT

An article and method for inhibiting the growth of microbes in biological and physiological fluids. The article has a support structure and derivatized particles that have an attached metal-ion sequestrant for inhibiting the growth of the microbes.

### CROSS REFERENCE TO RELATED APPLICATIONS

Reference is made to commonly assigned U.S. patent application Ser. No. ______ filed herewith entitled ARTICLE FOR INHIBITING MICROBIAL GROWTH by Joseph F. Bringley, David L. Patton, Richard W. Wien, Yannick J. F. Lerat (docket 87834), U.S. patent application Ser. No. ______ filed herewith entitled CONTAINER FOR INHIBITING MICROBIAL GROWTH IN LIQUID NUTRIENTS by David L. Patton, Joseph F. Bringley, Richard W. Wien, John M. Pochan, Yannick J. F. Lerat (docket 87472); U.S. patent application Ser. No. ______ filed herewith entitled USE OF DERIVATIZED NANOPARTICLES TO MINIMIZE GROWTH OF MICRO-ORGANISMS IN HOT FILLED DRINKS by Richard W. Wien, David L. Patton, Joseph F. Bringley, Yannick J. F. Lerat (docket 87471); U.S. patent application Ser. No. ______ filed herewith entitled DERIVATIZED NANOPARTICLES COMPRISING METAL-ION SEQUESTRAINT by Joseph F. Bringley (docket 87428); and U.S. patent application Ser. No. ______ filed herewith entitled COMPOSITION OF MATTER COMPRISING POLYMER AND DERIVATIZED NANOPARTICLES by Joseph F. Bringley, Richard W. Wien, Richard L. Parton (docket 87708), U.S. patent application Ser. No. ______ filed herewith entitled COMPOSITION COMPRISING INTERCALATED METAL-ION SEQUESTRANTS by Joseph F. Bringley, David L. Patton, Richard W. Wien (docket 87765), the disclosures of which are incorporated herein by reference.

### FIELD OF THE INVENTION

The present invention relates to an article for inhibiting the growth of micro-organisms in biological and physiological fluids and is capable of removing metal-ions from biological and physiological fluids and the exudates of wounds.

### BACKGROUND OF THE INVENTION

In recent years people have become very concerned about exposure to the hazards of microbe contamination. For example, exposure to certain strains of Escherichia coli through the ingestion of under-cooked beef can have fatal consequences. Exposure to Salmonella enteritidis through contact with unwashed poultry can cause severe nausea. Mold and yeast (Candida albicans) may cause skin infections. There is, in addition, increasing concern over pathogens, such as Salmonella and E. coli:O: 157, present in medical environments and concern over viruses such as Influenza, SARS, AIDS, and hepatitis. Indeed, some forms of bacteria, including Staphylococcus aureus are resistant to all but a few or one known antibiotic.

Noble metal-ions such as silver and gold ions are known for their antimicrobial properties and have been used in medical care for many years to prevent and treat infection. In recent years, this technology has been applied to consumer products to prevent the transmission of infectious disease and to kill harmful bacteria such as Staphylococcus aureus and Salmonella. In common practice, noble metals, metal-ions, metal salts or compounds containing metal-ions having antimicrobial properties, and other antimicrobial materials such as chlorophenol compounds (Triclosan™), isothiazolone (Kathon™), antibiotics, and some polymeric materials, may be applied to surfaces to impart an antimicrobial property to the surface. If, or when, the surface is inoculated with harmful microbes, the antimicrobial metal-ions or metal complexes, if present in effective concentrations, will slow or even prevent altogether the growth of those microbes. In addition, such compounds can be formed into, or coated upon, articles such as bandages, wound dressings, casts, personal hygiene items, etc.

In order for an antimicrobial article to be effective against harmful micro-organisms, the antimicrobial compound must come in direct contact with micro-organisms present in the surrounding environment, such as food, liquid nutrient or biological fluid. Since physiological fluids are often extraordinarily complex, the treatment of a multitude of microbial contaminants may be difficult, if not impossible, with one antimicrobial compound. Further, the antimicrobial ions or compounds may be precipitated or complexed by components of the biological or physiological fluids and rendered ineffective. Still further, micro-organisms such as bacteria may develop resistance to antibiotics, biocides and antimicrobials, and more dangerous microbes may result.

It has been recognized that small concentrations of metal-ions may play an important role in biological processes. For example, Mn, Fe, Ca, Zn, Cu and Al are essential bio-metals, and are required for most, if not all, living systems. Metal-ions play a crucial role in oxygen transport in living systems, and regulate the function of genes and replication in many cellular systems. Calcium is an important structural element in the formation of bones and other hard tissues. Mn, Cu and Fe are involved in metabolism and enzymatic processes. At high concentrations, metals may become toxic to living systems and the organism may experience disease or illness if the level cannot be controlled. As a result, the availability and concentrations of metal-ions in aqueous and biological environments is a major factor in determining the abundance, growth-rate and health of plant, animal and micro-organism populations.

It has been recognized that iron is an essential biological element, and that all living organisms require iron for survival and replication. Although the occurrence and concentration of iron is relatively high on the earth's surface, the availability of “free” iron is severely limited by the extreme insolubility of iron in aqueous environments. As a result, many organisms have developed complex methods of procuring “free” iron for survival and replication; and depend directly upon these mechanisms for their survival.

U.S. Pat. No. 5,217,998 to Hedlund et al. describes a method for scavenging free iron or aluminum in fluids such as physiological fluids by providing in such fluids a soluble polymer substrate having a chelator immobilized thereon. A composition is described which comprises a water-soluble conjugate comprising a pharmaceutically acceptable water-soluble polysaccharide covalently bonded to deferoxamine, a known iron chelator. The conjugate is said to be capable of reducing iron concentrations in body fluids in vivo.

U.S. Pat. No. 6,156,234 to Meyer-Ingold et al. describes novel wound coverings, which can remove interfering factors (such as iron ions) from the wound fluid of chronic wounds. The wound coverings may comprise iron chelators covalently bonded to a substrate such as cloth or cotton bandages.

U.S. Patent application 2003/0078209 A1 to Schmidt et al. describes solid porous compositions, substantially insoluble in water, comprising at least 25% by weight of an oxidized cellulose and having a significant capacity to bind iron. The invention also provides a method of sequestering dissolved iron from aqueous environments. The compositions may be used for the prevention or treatment of infections by bacteria or yeast.

There is a problem in that the above compositions are expensive to manufacture and do not bind metal-ions, such as iron, very strongly. There is a further problem in that the compositions above are difficult to apply to surfaces other than those specified, and are difficult to render transparent once applied to a surface.

Articles, such as bandages, personal hygiene items, and medical instruments, are needed that are able to provide for the general safety and health of the public. Articles are needed to protect the public from the spread of infectious disease and to prevent microbial contamination in health care environments. Materials and methods are needed to prepare articles having antimicrobial properties that are less, or not, susceptible to microbial resistance. Methods are needed that are able to target and remove specific, biologically important metal-ions, while leaving intact the concentrations of beneficial metal-ions.

### SUMMARY OF THE INVENTION

In accordance with one aspect of the present invention, there is provided an article for inhibiting the growth of microbes in biological and physiological fluids, the article having a support structure and comprising derivatized particles having an attached metal-ion sequestrant for inhibiting the growth of the microbes, wherein the derivatized particles have a stability constant greater than 10¹⁰ with iron (III).

In accordance with another aspect of the present invention, there is provided a method for inhibiting growth of microbes in biological and physiological fluids, comprising the steps of;
a. providing an article having a support structure and derivatized particles having an attached metal-ion sequestrant for inhibiting the growth of the microbes, wherein the derivatized particles have a stability constant greater than 10¹⁰ with iron (III); and
b. placing the article in contact with the biological and/or the physiological fluid so that the growth of microbes is inhibited in the biological and/or the physiological fluid.

These and other aspects, objects, features and advantages of the present invention will be more clearly understood and appreciated from a review of the following detailed description of the preferred embodiments and appended claims and by reference to the accompanying drawings.

### BRIEF DESCRIPTION OF THE DRAWINGS

In the detailed description of the preferred embodiments of the invention presented below, reference is made to the accompanying drawings in which:

FIG. 1 illustrates a plan view of a bandage made in accordance with the prior art as applied to the arm of an individual;

FIG. 2 is an enlarged partial cross sectional view of a portion of the bandage of FIG. 1 as taken along line 2-2;

FIG. 3 is a greatly enlarged partial cross sectional view of a portion of the bandage of FIG. 2 identified by circle 3;

FIG. 4 is an enlarged partial cross sectional view of a portion of the bandage of FIG. 1 similar to FIG. 2, but made in accordance with the present invention;

FIG. 5 is a greatly enlarged partial cross sectional view of a portion of the bandage of FIG. 4 identified by circle 5;

FIG. 6 is an enlarged partial cross sectional view similar to FIG. 5 of a portion of modified bandage also made in accordance with the present invention;

FIG. 7 is a perspective view of a tampon made in accordance with the present invention partially broken away to illustrate an inner core;

FIG. 8 is an enlarged partial cross sectional view of a portion of the tampon of FIG. 7 as taken along line 8-8;

FIG. 9 is a perspective view of a sanitary napkin for use by woman also made in accordance with the present invention;

FIG. 10 is an enlarged partial cross sectional view of a portion of the sanitary napkin of FIG. 9 as taken along line 10-10;

FIG. 11 illustrates an exploded perspective view of a disposable diaper made in accordance with the present invention; and

FIG. 12 is an enlarged partial cross sectional view of a portion of the diaper of FIG. 11 as taken along line 12-12.

### DETAILED DESCRIPTION OF THE INVENTION

The articles of the invention may comprise health care items such as band-aids, bandages, and wound healing items, and personal care items such as diapers, tampons, feminine napkins, gauze and cotton and other articles. The articles of the invention are useful for preventing microbial growth in biological and physiological fluids. The articles of the invention may provide for the health and safety of the general public. The articles of the invention may also provide for the health and safety of animals. The articles of the invention do not release chemicals that can be harmful to humans or that may leach into aquatic or surrounding environments, and are cleaner and safer in preventing microbial contamination and infectious disease. The articles of the invention are able to remove or sequester metal-ions such as Zn, Cu, Mn and Fe which are essential for biological growth, and thus may inhibit the growth of harmful micro-organisms such as bacteria, viruses, and fungi in physiological fluids within or upon the user of said article. The articles of the invention when placed in contact with physiological fluids, removes essential biological metal-ions, and thus “starves” the micro-organisms present in such fluids of minute quantities of essential nutrients (metal-ions) and limits their growth; thereby reducing the risk due to bacterial, viral and other infectious diseases.

The invention provides an article for inhibiting the growth of a microbes in biological and physiological fluids, said article having a support structure and comprising derivatized particles having an attached metal-ion sequestrant for inhibiting the growth of said microbes, wherein the derivatized particles have a stability constant greater than 10¹⁰ with iron (III). It is further preferred that said derivatized particles have a stability constant greater than 10²⁰ with iron (III). This is preferred because iron is an essential metal-ion nutrient for virtually all micro-organisms. The term stability constant will be defined in detail below. It is preferred that said support structure is made of fibers, fabric, textiles, plastic or paper.

The derivatized particles of the invention have an attached metal-ion sequestrant. The metal-ion sequestrants are attached to particles in order to “anchor” them in place and prevent the diffusion of the sequestrant. In this manner, metal-ions chelated or complexed by the sequestrant are thereby “anchored” to the particles and may not diffuse away. It is preferred that the derivatized particles are further immobilized on a support structure. This is preferred because the derivatized particles may then sequester metal-ions within the support structure. Particles suitable for practice of the invention are inorganic or organic particles. Inorganic particles include colloids and other particulates such as silica oxides, alumina oxides, boehmites, titanium oxides, zinc oxides, tin oxides, zirconium oxides, yttrium oxides, hafnium oxides, clays or alumina silicates, and more preferably comprise silicon dioxide, alumina oxide, clays or boehmite. The term “clay” is used to describe silicates and alumino-silicates, and derivatives thereof. Some examples of clays, which are commercially available, are montmorillonite, bentonite, hectorite, and synthetic derivatives such as laponite. Other examples include hydrotalcites, zeolites, alumino-silicates, and metal (oxy)hydroxides given by the general formula, MₐO_{b}(OH)_{c}, where M is a metal-ion and a, b and c are integers. Organic particles include latexes and ion exchange resins.

The articles made in accordance with the invention comprises a derivatized particle having an attached metal-ion sequestrant having a high-affinity for metal-ions. It is preferred that the metal-ion sequestrant has a high-affinity for biologically important metal-ions such as Mn, Zn, Cu and Fe. It is further preferred that the metal-ion sequestering agent has a high-selectivity for biologically important metal-ions such as Mn, Zn, Cu and Fe. In a particular embodiment, it is preferred that said derivatized particles are immobilized on the support structure and have a high-affinity for biologically important metal-ions such as Mn, Zn, Cu and Fe. It is further preferred that said derivatized particles are immobilized on the support structure and have a high-selectivity for biologically important metal-ions such as Mn, Zn, Cu and Fe. It is still further preferred that said derivatized particles are immobilized on the support structure and have a stability constant for iron greater than 10²⁰, more preferably greater than 10³⁰.

A measure of the “affinity” of metal-ion sequestrants for various metal-ions is given by the stability constant (also often referred to as critical stability constants, complex formation constants, equilibrium constants, or formation constants) of that sequestrant for a given metal-ion. Stability constants are discussed at length in “Critical Stability Constants”, A. E. Martell and R. M. Smith, Vols. 1-4, Plenum, N.Y. (1977), “Inorganic Chemistry in Biology and Medicine”, Chapter 17, ACS Symposium Series, Washington, D.C. (1980), and by R. D. Hancock and A. E. Martell, Chem. Rev. vol. 89, p. 1875-1914 (1989). The ability of a specific molecule or ligand to sequester a metal-ion may depend also upon the pH, the concentrations of interfering ions, and the rate of complex formation (kinetics). Generally, however, the greater the stability constant, the greater the binding affinity for that particular metal-ion. Often the stability constants are expressed as the natural logarithm of the stability constant. Herein the stability constant for the reaction of a metal-ion (M) and a sequestrant or ligand (L) is defined as follows:
M+n L⇄MLₙ

where the stability constant is βₙ=[MLₙ]/[M][L]ⁿ, wherein [MLₙ] is the concentration of “complexed” metal-ion, [M] is the concentration of free (uncomplexed) metal-ion and [L] is the concentration of free ligand. The log of the stability constant is log βₙ, and n is the number of ligands, which coordinate with the metal. It follows from the above equation that if βₙ is very large, the concentration of “free” metal-ion will be very low. Ligands with a high stability constant (or affinity) generally have a stability constant greater than 10¹⁰ or a log stability constant greater than 10 for the target metal. Preferably the ligands have a stability constant greater than 10¹⁵ for the target metal-ion. Table 1 lists common ligands (or sequestrants) and the natural logarithm of their stability constants (log βₙ) for selected metal-ions.

TABLE 1
Common ligands (or sequestrants) and the natural logarithm
of their stability constants (log β) for selected metal-ions.

| Ligand | Ca | Mg | Cu(II) | Fe(III) | Al | Ag | Zn |
|---|---|---|---|---|---|---|---|
| alpha-amino | | | | | | | |
| carboxylates | | | | | | | |
| EDTA | 10.6 | 8.8 | 18.7 | 25.1 | | 7.2 | 16.4 |
| DTPA | 10.8 | 9.3 | 21.4 | 28.0 | 18.7 | 8.1 | 15.1 |
| CDTA | 13.2 | 21.9 | 30.0 | | | | |
| NTA | | | | 24.3 | | | |
| DPTA | 6.7 | 5.3 | 17.2 | 20.1 | 18.7 | 5.3 | |
| PDTA | 7.3 | | 18.8 | | | | 15.2 |
| citric Acid | 3.50 | 3.37 | 5.9 | 11.5 | 7.98 | 9.9 | |
| salicylic acid | | | | 35.3 | | | |
| Hydroxamates | | | | | | | |
| Desferroxamine B | | | | 30.6 | | | |
| acetohydroxamic | | | | 28 | | | |
| acid | | | | | | | |
| Catechols | | | | | | | |
| 1,8-dihydroxy | | | | 37 | | | |
| naphthalene | | | | | | | |
| 3,6 sulfonic acid | | | | | | | |
| MECAMS | | | | 44 | | | |
| 4-LICAMS | | | | 27.4 | | | |
| 3,4-LICAMS | 16.2 | | | 43 | | | |
| 8-hydroxyquinoline | | | | 36.9 | | | |
| disulfocatechol | 5.8 | 6.9 | 14.3 | 20.4 | 16.6 | | |

EDTA is ethylenediamine tetraacetic acid and salts thereof, DTPA is diethylenetriaminepentaacetic acid and salts thereof, DPTA is Hydroxylpropylenediaminetetraacetic acid and salts thereof, NTA is nitrilotriacetic acid and salts thereof, CDTA is 1,2-cyclohexanediamine tetraacetic acid and salts thereof, PDTA is propylenediammine tetraacetic acid and salts thereof. Desferroxamine B is a commercially available iron chelating drug, desferal®. MECAMS, 4-LICAMS and 3,4-LICAMS are described by Raymond et al. in “Inorganic Chemistry in Biology and Medicine”, Chapter 18, ACS Symposium Series, Washington, D.C. (1980). Log stability constants are from “Critical Stability Constants”, A. E. Martell and R. M. Smith, Vols. 1-4, Plenum Press, NY (1977); “Inorganic Chemistry in Biology and Medicine”, Chapter 17, ACS Symposium Series, Washington, D.C. (1980); R. D. Hancock and A. E. Martell, Chem. Rev. vol. 89, p. 1875-1914 (1989) and “Stability Constants of Metal-ion Complexes”, The Chemical Society, London, 1964.

In many instances, the growth of a particular micro-organism may be limited by the availability of a particular metal-ion, for example, due to a deficiency of this metal-ion. In such cases, it is desirable to select a metal-ion sequestrant with a very high specificity or selectivity for a given metal-ion. Metal-ion sequestrants of this nature may be used to control the concentration of the target metal-ion and thus limit the growth of the organism(s), which require this metal-ion. However, it may be necessary to control the concentration of the target metal, without affecting the concentrations of beneficial metal-ions such as potassium and calcium. One skilled in the art may select a metal-ion sequestrant having a high selectivity for the target metal-ion. The selectivity of a metal-ion sequestrant for a target metal-ion is given by the difference between the log of the stability constant for the target metal-ion, and the log of the stability constant for the interfering (beneficial) metal-ions. For example, if a treatment required the removal of Fe(III), but it was necessary to leave the Ca-concentration unaltered, then from Table 1, DTPA would be a suitable choice since the difference between the log stability constants 28−10.8=17.2, is very large. 3,4-LICAMS would be a still more suitable choice since the difference between the log stability constants 43−16.2=26.8, is the largest in Table 1.

It is preferred that said metal-ion sequestrant has a high-affinity for iron, and in particular iron (III). It is preferred that the stability constant of the sequestrant for iron (III) be greater than 10¹⁰. It is still further preferred that the metal-ion sequestrant has a stability constant for iron greater than 10²⁰. It is still further preferred that the metal-ion sequestrant has a stability constant for iron greater than 10³⁰.

In a preferred embodiment, the derivatized particles comprise derivatized nanoparticles comprising inorganic nanoparticles having an attached metal-ion sequestrant, wherein said inorganic nanoparticles have an average particle size of less than 200 nm and the derivatized nanoparticles have a stability constant greater than 10¹⁰ with iron (III). It is further preferred that the derivatized nanoparticles have a stability constant greater than 10²⁰ with iron (III). The derivatized nanoparticles are preferred because they have very high surface area and may have a very high-affinity for the target metal-ions. It is preferred that the nanoparticles have an average particle size of less than 100 nm. It is further preferred that the nanoparticles have an average size of less than 50 nm, and most preferably less than 20 nm. Preferably greater than 95% by weight of the nanoparticles are less than 200 nm, more preferably less than 100 nm, and most preferably less than 50 nm. This is preferred because as the particle size becomes smaller, the particles scatter visible-light less strongly. Therefore, the derivatized nanoparticles can be applied to clear, transparent surfaces without causing a hazy or a cloudy appearance at the surface. This allows the particles of the present invention to be applied to articles without changing the appearance of the article. It is preferred that the nanoparticles have a very high surface area, since this provides more surface with which to covalently bind the metal-ion sequestrant, thus improving the capacity of the derivatized nanoparticles for binding metal-ions. It is preferred that the nanoparticles have a specific surface area of greater than 100 m²/g, more preferably greater than 200 m²/g, and most preferably greater than 300 m²/g. For applications of the invention in which the concentrations of contaminant or targeted metal-ions in the environment is high, it is preferred that the nanoparticles have a particle size of less than 20 nm and a surface area of greater than 300 m²/g. Derivatized nanoparticles are described at length in U.S. patent application Ser. No. ______ filed herewith entitled DERIVATIZED NANOPARTICLES COMPRISING METAL-ION SEQUESTRAINT by Joseph F. Bringley (docket 87428).

The inorganic nanoparticles of the invention preferably comprise silica oxides, alumina oxides, boehmites, titanium oxides, zinc oxides, tin oxides, zirconium oxides, yttrium oxides, hafnium oxides, clays or alumina silicates, and more preferably comprise silicon dioxide, alumina oxide, clays or boehmite. The nanoparticles may comprise a combination or mixture of the above materials. The term “clay” is used to describe silicates and alumino-silicates, and derivatives thereof. Some examples of clays which are commercially available are montmorillonite, hectorite, and synthetic derivatives such as laponite. Other examples include hydrotalcites, zeolites, alumino-silicates, and metal (oxy)hydroxides given by the general formula, MₐO_{b}(OH)_{c}, where M is a metal-ion and a, b and c are integers.

It is preferred that the derivatized nanoparticles have a high stability constant for the target metal-ion(s). The stability constant for the derivatized nanoparticle will largely be determined by the stability constant for the attached metal-ion sequestrant. However, the stability constant for the derivatized nanoparticles may vary somewhat from that of the attached metal-ion sequestrant. Generally, it is anticipated that metal-ion sequestrants with high stability constants will give derivatized nanoparticles with high stability constants. For a particular application, it may be desirable to have a derivatized nanoparticle with a high selectivity for a particular metal-ion. In most cases, the derivatized nanoparticle will have a high selectivity for a particular metal-ion if the stability constant for that metal-ion is about 10⁶ greater than for other ions present in the system.

Metal-ion sequestrants may be chosen from various organic molecules. Such molecules having the ability to form complexes with metal-ions are often referred to as “chelators”, “complexing agents”, and “ligands”. Certain types of organic functional groups are known to be strong “chelators” or sequestrants of metal-ions. It is preferred that the sequestrants of the invention contain alpha-amino carboxylates, hydroxamates, or catechol, functional groups.

Hydroxamates, or catechol, functional groups are preferred. Alpha-amino carboxylates have the general formula:
R—[N(CH₂CO₂M)—(CH₂)ₙ—N(CH₂CO₂M)₂]ₓ
where R is an organic group such as an alkyl or aryl group; M is H, or an alkali or alkaline earth metal such as Na, K, Ca or Mg, or Zn; n is an integer from 1 to 6; and x is an integer from 1 to 3. Examples of metal-ion sequestrants containing alpha-amino carboxylate functional groups include ethylenediaminetetraacetic acid (EDTA), ethylenediaminetetraacetic acid disodium salt, diethylenetriaminepentaacetic acid (DTPA), Hydroxylpropylenediaminetetraacetic acid (DPTA), nitrilotriacetic acid, triethylenetetraaminehexaacetic acid, N,N-bis(o-hydroxybenzyl) ethylenediamine-N,N′ diacteic acid, and ethylenebis-N,N′-(2-o-hydroxyphenyl)glycine.

Hydroxamates (or often called hydroxamic acids) have the general formula: 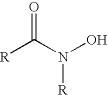
where R is an organic group such as an alkyl or aryl group. Examples of metal-ion sequestrants containing hydroxamate functional groups include acetohydroxamic acid, benzohydroxamic acid and desferroxamine B, the iron chelating drug desferal.

Catechols have the general formula: 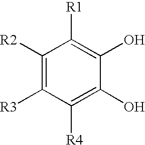
Where R1, R2, R3 and R4 may be H, an organic group such as an alkyl or aryl group, or a carboxylate or sulfonate group. Examples of metal-ion sequestrants containing catechol functional groups include catechol, disulfocatechol, dimethyl-2,3-dihydroxybenzamide, mesitylene catecholamide (MECAM) and derivatives thereof, 1,8-dihydroxynaphthalene-3,6-sulfonic acid, and 2,3-dihydroxynaphthalene-6-sulfonic acid.

In a preferred embodiment of the invention, the metal-ion sequestrant is attached to the particle, by reaction of the particle with a metal alkoxide intermediate of the sequestrant having the general formula.
M(OR)₄₋ₓR′ₓ;
wherein M is silicon, titanium, aluminum, tin, or germanium;
x is an integer from 1 to 3;
R is an organic group; and
R′ is an organic group containing an alpha-amino carboxylate, a hydroxamate, or a catechol, functional group. It is further preferred that R′ is an organic group containing a hydroxamate, or a catechol, functional group.

In a preferred embodiment the metal-ion sequestrant is attached to a particle by reaction of the particle with a silicon alkoxide intermediate having the general formula:
Si(OR)₄₋ₓR′ₓ;
wherein x is an integer from 1 to 3;
R is an alkyl group; and
   R′ is an organic group containing an alpha amino carboxylate, a hydroxamate, or a catechol. The —OR-group attaches the silicon alkoxide to the core particle surface via a hydrolysis reaction with the surface of the particles. Materials suitable for practice of the invention include N-(trimethoxysilylpropyl)ethylenediamine triacetic acid, trisodium salt, N-(triethoxysilylpropyl)ethylenediamine triacetic acid, trisodium salt, N-(trimethoxysilylpropyl)ethylenediamine triacetic acid, N-(trimethoxysilylpropyl)diethylenetriamine tetra acetic acid, N-(trimethoxysilylpropyl)amine diacetic acid, and metal-ion salts thereof.

It is preferred that substantially all (greater than 90%) of the metal-ion sequestrant is covalently bound to the particles, and is thus “anchored” to the particle. Metal-ion sequestrant that is not bound to the particles may dissolve and quickly diffuse through a system; and may be ineffective in removing metal-ions from the system. It is further preferred that the metal-ion sequestrant is present in an amount sufficient, or less than sufficient, to cover the surfaces of all particles. This is preferred because it maximizes the number of covalently bound metal-ion sequestrants, since once the surface of the particles is covered, no more covalent linkages to the particle may result.

It is preferred that the article(s) of the invention comprise a polymer, or polymeric layer containing said derivatized particles. The article may comprise the polymer itself containing said derivatized particles, or alternatively, the derivatized particles may be contained with a polymeric layer attached to a support structure. It is preferred that said polymer is permeable to water. It is important that the polymer is permeable to water because permeability facilitates the contact of the target metal-ions with the metal-ion sequestrant, which, in turn, facilitates the sequestration of the metal-ions within the polymer or polymeric layer. A measure of the permeability of various polymeric addenda to water is given by the permeability coefficient, P which is given by
P=(quantity of permeate)(film thickness)/[area×time×(pressure drop across the film)]
Permeability coefficients and diffusion data of water for various polymers are discussed by J. Comyn, in Polymer Permeability, Elsevier, N.Y., 1985 and in “Permeability and Other Film Properties Of Plastics and Elastomers”, Plastics Design Library, NY, 1995. The higher the permeability coefficient, the greater the water permeability of the polymeric media. The permeability coefficient of a particular polymer may vary depending upon the density, crystallinity, molecular weight, degree of cross-linking, and the presence of addenda such as coating-aids, plasticizers, etc. It is preferred that the polymer has a water permeability of greater than 1000 [(cm³ cm)/(cm²sec/Pa)]×10¹³.

It is further preferred that the polymer has a water permeability of greater than 5000 [(cm³ cm)/(cm²sec/Pa)]×10¹³. Preferred polymers for practice of the invention are polyvinyl alcohol, cellophane, water-based polyurethanes, polyester, nylon, high nitrile resins, polyethylene-polyvinyl alcohol copolymer, polystyrene, ethyl cellulose, cellulose acetate, cellulose nitrate, aqueous latexes, polyacrylic acid, polystyrene sulfonate, polyamide, polymethacrylate, polyethylene terephthalate, polystyrene, polyethylene, polypropylene or polyacrylonitrile. It is preferred that the metal-ion sequestrant comprises 0.1 to 50.0% by weight of the polymer, and more preferably 1% to 10% by weight of the polymer.

In a preferred embodiment, the article(s) of the invention may further comprise a barrier layer, wherein the polymeric layer is between the surface of the article and the barrier layer and wherein the barrier layer does not contain the derivatized particles. It is preferred that the barrier layer is permeable to water, and has a thickness preferably in the range of 0.1 to 10.0 microns. It is preferred that microbes cannot pass or diffuse through the barrier layer. The barrier layer may provide several functions including improving the physical strength and toughness of the article and resistance to scratching, marring, cracking, etc. However, the primary purpose of the barrier layer is to provide a barrier through which micro-organisms cannot pass. It is important to limit, or eliminate, the direct contact of micro-organisms with the metal-ion sequestrant or the layer containing the metal-ion sequestrant, since many micro-organisms, under conditions of iron deficiency, may bio-synthesize molecules which are strong chelators for iron, and other metals. These bio-synthetic molecules are called “siderophores” and their primary purpose is to procure iron for the micro-organisms. Thus, if the micro-organisms are allowed to directly contact the metal-ion sequestrant, they may find a rich source of iron there, and begin to colonize directly at these surfaces. The siderophores produced by the micro-organisms may compete with the metal-ion sequestrant for the iron (or other bio-essential metal) at their surfaces. The barrier layer of the invention does not contain the derivatized particles, and because micro-organisms are large, they may not pass or diffuse through the barrier layer. The barrier layer thus prevents contact of the micro-organisms with the polymeric layer containing the metal-ion sequestrant of the invention. Materials suitable for barrier layers are described at length in U.S. patent application Ser. No. ______ filed herewith entitled COMPOSITION OF MATTER COMPRISING POLYMER AND DERIVATIZED NANOPARTICLES by Joseph F. Bringley et al. (docket 87708).

The articles of the invention are useful for preventing microbial growth in biological and physiological fluids, and may be used to treat or prevent infection in wounds, and to prevent infection resulting from contact with physiological fluids such as blood, urine, fecal matter, etc. In a preferred embodiment, the article is designed to be placed against the skin of an individual. In another preferred embodiment, the article comprises a bandage. It is preferred that the bandage includes a liquid permeable barrier layer for allowing said biological or physiological fluids to come in contact with the derivatized particles. In a preferred embodiment, the article comprises a diaper. It is preferred that said diaper includes a liquid permeable membrane for allowing said biological or physiological fluids to come in contact with the derivatized particles.

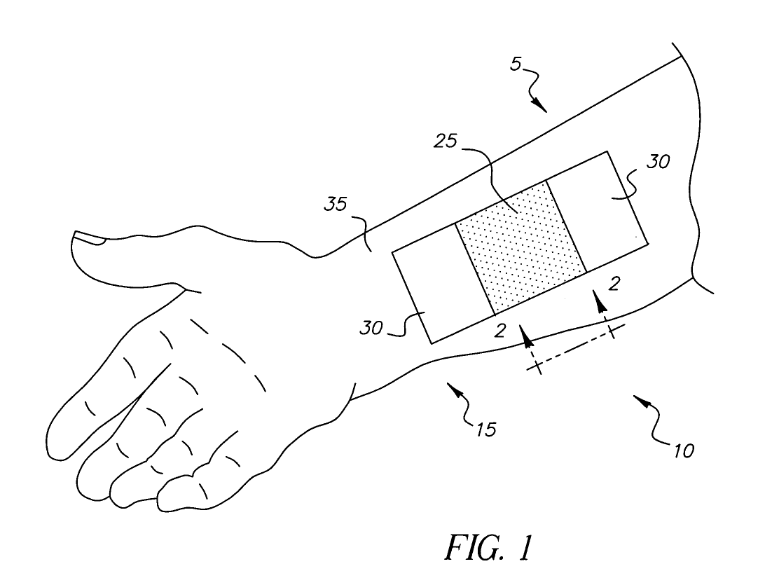

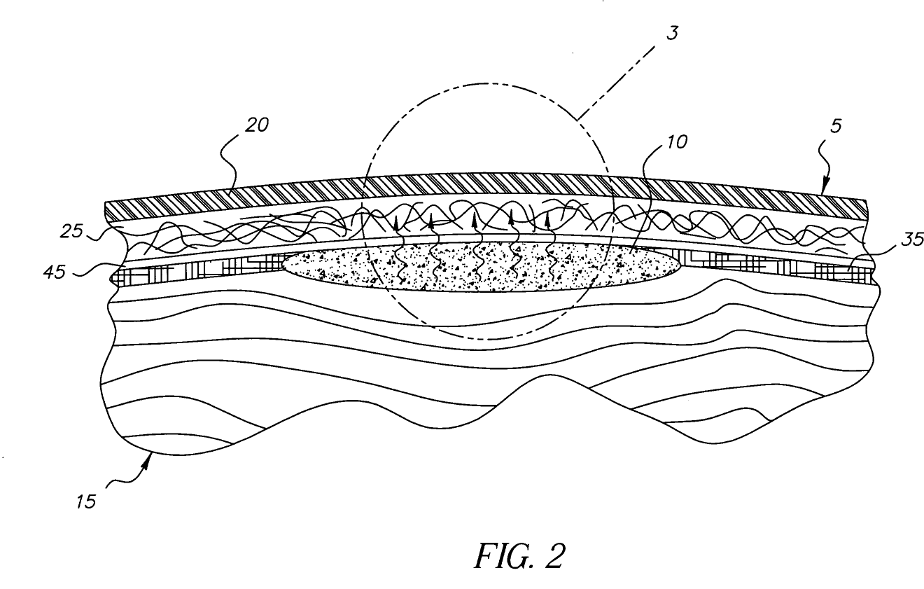

Referring to FIGS. 1 and 2, there is illustrated a cross-sectional view of a typical prior art article such as a bandage 5 placed over a wound 10 on an arm 15 of an individual. In the embodiment illustrated, the bandage 5 comprises a support 20 holding a pad 25 for absorbing biological and physiological fluids and the exudates of wounds. The support 20 also holds the adhesive section 30 for attaching the bandage 5 to the skin 35. The pad 25 may be covered with an anti stick layer 45 to prevent the pad 25 from sticking to the wound 10.

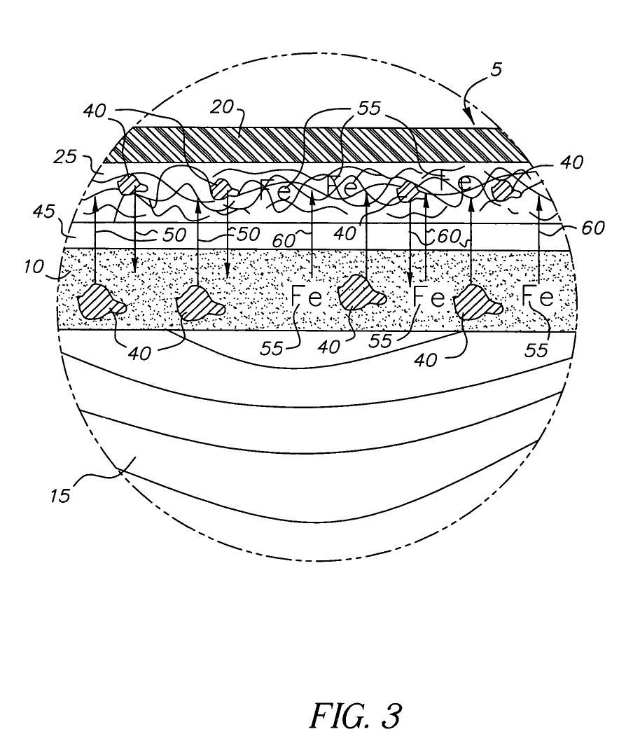

Referring now to FIG. 3, there is illustrated an enlarged partial cross sectional view of a portion of the bandage of FIGS. 1 and 2 identified by circle 3. The micro-organisms 40 are free to move from the wound 10 through the non-stick layer 45 of the bandage 5 and back to the wound 10 as indicated by the arrows 50. Likewise the “free” iron 55 is free to move from the wound 10 through the non-stick layer 45 of the bandage 5 and back to the wound 10 as indicated by the arrows 60.

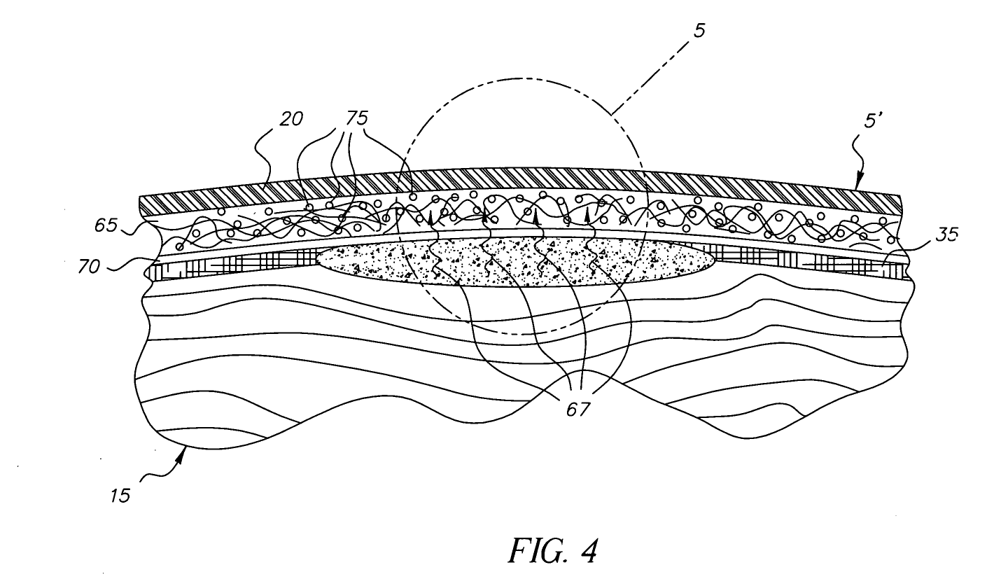

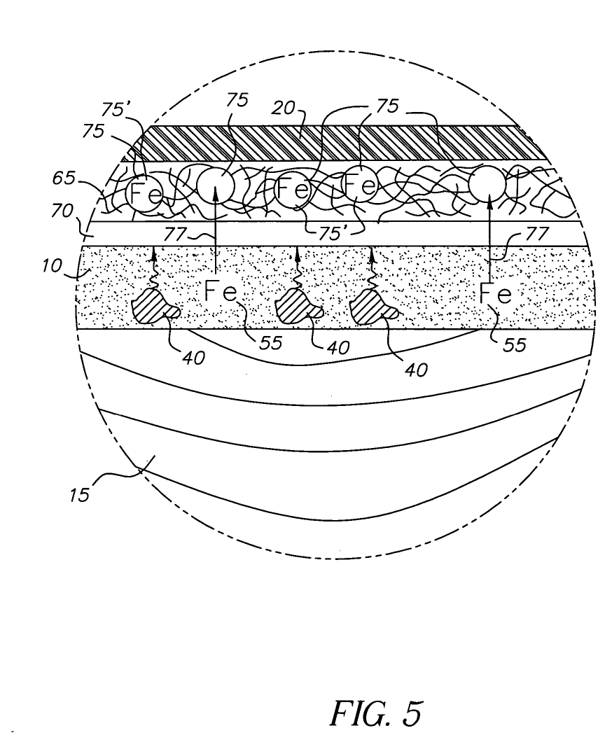

Referring to FIGS. 4 and 5, there is illustrated an embodiment of the article such as the bandage 5′ made in accordance with the present invention. The bandage 5′ of FIGS. 4 and 5 is similar to the bandage 5 of FIGS. 1-3, like numerals indicating like parts and operation. The bandage 5′ comprises a support 20 holding a pad 65 for absorbing biological and physiological fluids and the exudates of wounds as indicated by the arrows 67. The support 20 also holds the adhesive section 30 for attaching the bandage 5′ to the skin 35. The pad 65 is covered with an anti-stick barrier layer 70 to prevent the pad 25 from sticking to the wound 10. The pad 65 contains derivatized particles 75. The anti-stick barrier layer 70 preferably does not contain the derivatized particles 75. The primary purpose of the anti-stick barrier layer 70 is to provide a barrier through which micro-organisms 40 present in the biological and physiological fluids and the exudates of wounds cannot pass. It is important to limit or eliminate direct contact of micro-organisms 40 with the derivatized particles 75 or the layer containing the derivatized particles 75, since many micro-organisms 40, under conditions of iron deficiency, may bio-synthesize molecules which are strong chelators for iron, and other metals. These bio-synthetic molecules are called “siderophores” and their primary purpose is to procure iron for the micro-organisms 40. Thus, if the micro-organisms 40 are allowed to directly contact the derivatized particles 75, they may find a rich source of iron there, and begin to colonize causing infection. The siderophores produced by the micro-organisms may compete with the derivatized particles for the iron (or other bio-essential metal) at their surfaces. However, the energy required for the organisms to adapt their metabolism to synthesize these siderophores will impact significantly their growth rate. Thus, one object of the invention is to lower growth rate of organisms in the contained biological and physiological fluids and the exudates of wounds. Since the anti-stick barrier layer 70 of the invention does not contain the derivatized particles 75, and because micro-organisms are large, the micro-organisms may not pass or diffuse through the anti-stick layer 70. The anti-stick barrier layer 70 thus prevents contact of the micro-organisms with the pad 65 containing the derivatized particles 75 of the invention. It is preferred that the anti-stick barrier layer 70 is permeable to water. It is preferred that the barrier layer 70 has a thickness “x” in the range of 0.1 microns to 10.0 microns. It is preferred that microbes are unable to penetrate, to diffuse or pass through the anti-stick barrier layer 70. Derivatized particles 75 with a sequestered metal-ion is indicated by numeral 75′.

Referring again to FIG. 5, the enlarged sectioned view of the bandage 5′ shown in 4, illustrates a bandage having anti-stick barrier layer 70, which is in direct contact with the wound 10, the pad 65 containing the derivatized particles 75 and the outer support 20. However, the bandage of FIG. 2 comprises a pad 25 that does not contain derivatized particles. In the prior art bandage 5 illustrated in FIGS. 1, 2 and 3, the micro-organisms 40 are free to gather the “free” iron ions 55. In the example shown in FIGS. 4 and 5, the pad 65 contains immobilized derivatized particles 75 as provided by the derivatized particles of the invention. In order for the derivatized particles 75 to work properly, the pad 65 containing the derivatized particles 75 must be permeable to the biological and physiological fluids and the exudates of wounds. Preferred polymers for anti-stick barrier layer 70 of the invention are polyvinyl alcohol, cellophane, water-based polyurethanes, polyester, nylon, high nitrile resins, polyethylene-polyvinyl alcohol copolymer, polystyrene, ethyl cellulose, cellulose acetate, cellulose nitrate, aqueous latexes, polyacrylic acid, polystyrene sulfonate, polyamide, polymethacrylate, polyethylene terephthalate, polystyrene, polyethylene, polypropylene or polyacrylonitrile. A water permeable polymer permits water to move freely through the anti-stick barrier layer 70 allowing the “free” iron ion 55 to reach as indicated by the arrows 77 and be captured by the derivatized particles 75. The micro-organism 40 is too large to pass through the anti-stick barrier layer 70 so it cannot reach the sequestered iron ion 75′ now held by the derivatized particles 75. By using the derivatized particles 75 to significantly reduce the amount of “free” iron ions 55 in the biological and physiological fluids and the exudates of wounds, the growth of the micro-organism 40 is eliminated or severely reduced.

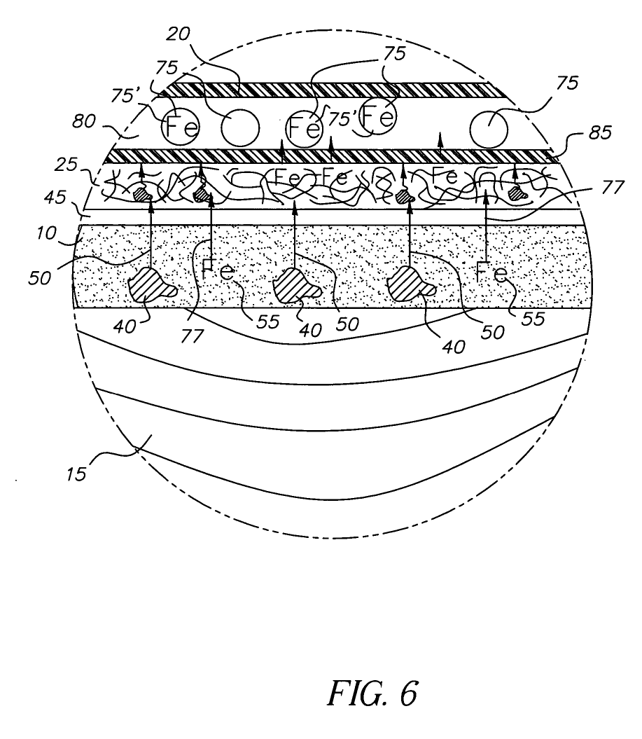

Referring to FIG. 6, there is illustrated another embodiment bandage 5″ that is similar to bandage 5′of FIG. 4, like numerals indicating like parts and operation. The derivatized particles 75 in bandage 5″ are immobilized in an inner polymer 80 located between the support 20 and an inner barrier layer 85. In order for the derivatized particles 75 to work properly, the inner polymer 80 containing the derivatized particles 75 must be permeable to water. Preferred polymers for layers 80 and 85 of the invention are polyvinyl alcohol, cellophane, water-based polyurethanes, polyester, nylon, high nitrile resins, polyethylene-polyvinyl alcohol copolymer, polystyrene, ethyl cellulose, cellulose acetate, cellulose nitrate, aqueous latexes, polyacrylic acid, polystyrene sulfonate, polyamide, polymethacrylate, polyethylene terephthalate, polystyrene, polyethylene, polypropylene or polyacrylonitrile. A water permeable polymer permits water to move freely through the polymer 80 allowing the “free” iron ion 55 to reach and be captured by the derivatized particles 75. An additional barrier 85 may be used to prevent the micro-organism 40 from reaching the inner polymer material 80 containing the derivatized particles 75. Like the inner polymer material 80, the inner barrier layer 85 must be made of a water permeable polymer as previously described. The micro-organism 40 is too large to pass through the barrier 85 or the polymer 80 so it cannot reach the sequestered iron ion 75′ now held by the derivatized particles 75. By using the derivatized particles 75 to significantly reduce the amount of “free” iron ions 55 in the biological and physiological fluids and the exudates of wounds captured by the pad 25, the growth of the micro-organism 75 is eliminated or severely reduced preventing infection of the wound 10.

In another preferred embodiment of the present invention, the article comprises gauze with the derivatized particles 75 incorporated therein as is shown by the pad 65 in FIG. 5.

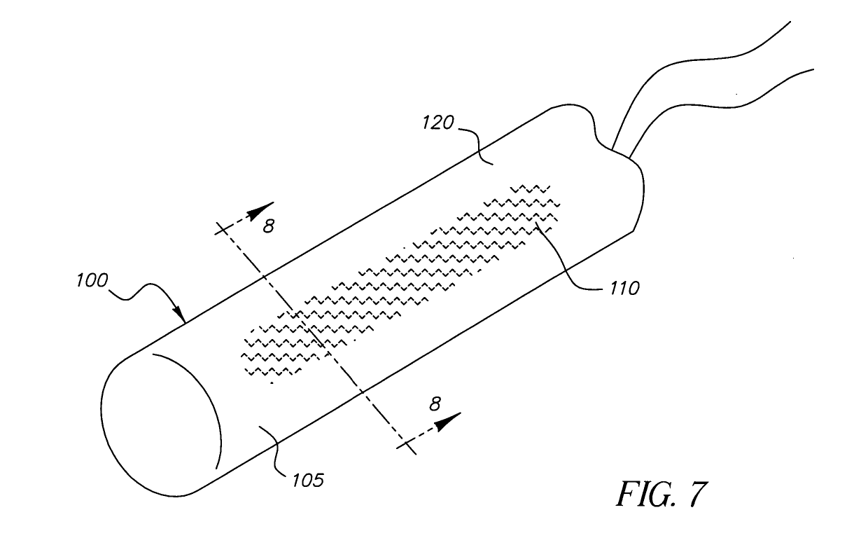

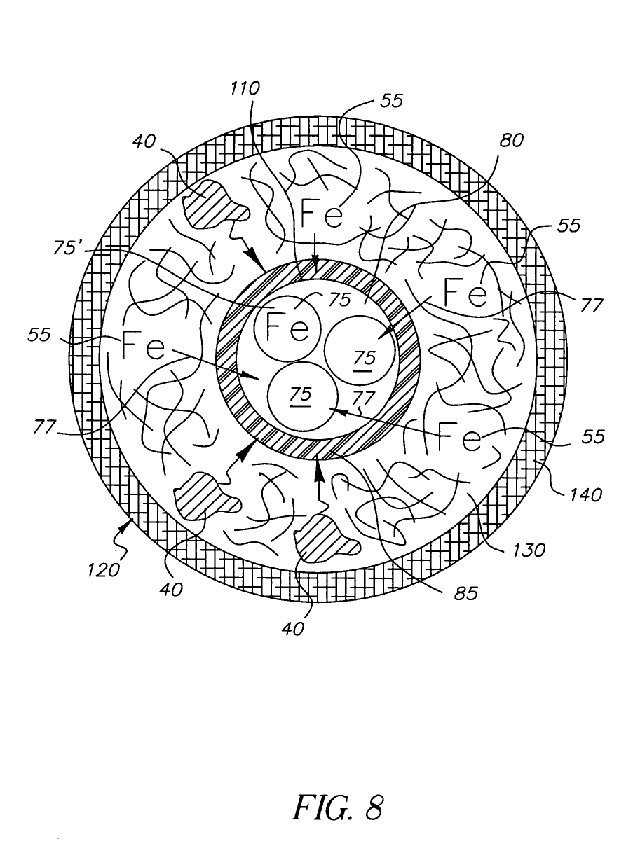

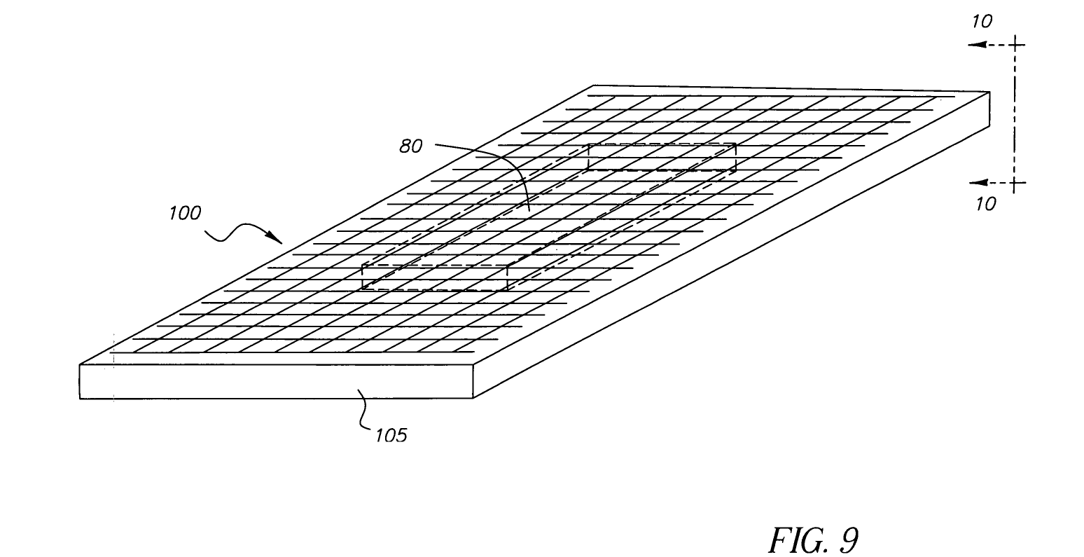

In another embodiment of the present invention, the article is designed to be placed within a living animal such as a human, and relates to fibrous articles intended for absorption of body fluids and, in particular, to tampons and similar catamenial devices. As shown in FIGS., 7, 8, 9 and 10, the fibrous absorbent article 100 comprises fibrous material 105 capable of absorbing body fluids such as catamenial fluids and the like. The fibrous material 105 may be arranged to form a woven or non-woven structure. The fibrous absorbent article 100 is, in the particular example of FIG. 7, a tampon 120 which has a well-known cylindrical shape and may consist of a number of fibrous layers as shown in FIG. 8. As another example, a sanitary napkin 150 as shown in FIG. 9 may form the absorbent article and may consist of a plurality of fibrous absorption fabrics. The tampon 120 made in accordance with the present invention has a center core 110 containing derivatized particles 75 capable of sequestering “free” iron ions 55.

Referring again to FIG. 8, there is illustrated an enlarged sectioned view of the tampon 120 shown in 7. The tampon consists of a number of fibrous layers, such as inner layer 130 and outer layer 140. The derivatized particles 75 are immobilized in an inner polymer 80 disposed or incorporated in the fibrous absorbent tampon 120 and may be surrounded by a barrier layer 85. In order for the derivatized particles 75 to work properly, the inner polymer 80 containing the derivatized particles 75 must be permeable to water. Preferred polymers for layers 80 and 85 of the invention have been previously described. A water permeable polymer permits water to move freely through the polymer 80 allowing the “free” iron ion 55 to reach and be captured by the agent 75. An additional barrier 85 maybe used to prevent the micro-organism 40 from reaching the inner polymer material 80 containing the derivatized particles 75. Like the inner polymer material 80, the inner barrier layer 85 must be made of a water permeable polymer as previously described. The micro-organism 40 is too large to pass through the barrier 85 or the polymer 80 so it cannot reach the sequestered iron ion 75′ now held by the derivatized particles 75. By using the derivatized particles 75 to significantly reduce the amount of “free” iron ions 55 in the catamenial fluids captured by the tampon 120, the growth of the micro-organism 75 is eliminated or severely reduced preventing infection.

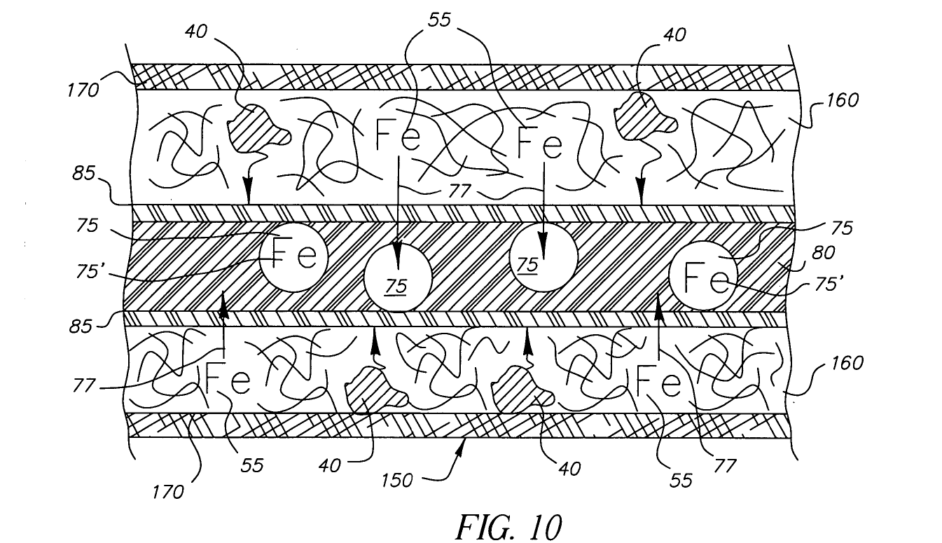

Referring to FIG. 10, there is illustrated an enlarged sectioned view of the sanitary napkin 150 shown in 9. The sanitary napkin 150 consists of a number of fibrous layers, such as inner layer 160 and outer layer 170. The derivatized particles 75 are immobilized in an inner polymer 80 disposed or incorporated in the fibrous absorbent sanitary napkin 150 and may be surrounded by a barrier layer 85. In order for the derivatized particles 75 to work properly, the inner polymer 80 containing the derivatized particles 75 must be permeable to water. Preferred polymers for layers 80 and 85 of the invention have been previously described. A water permeable polymer permits water to move freely through the polymer 80 allowing the “free” iron ion 55 to reach and be captured by the agent 75. An additional barrier 85 maybe used to prevent the micro-organism 40 from reaching the inner polymer material 80 containing the derivatized particles 75. Like the inner polymer material 80, the inner barrier layer 85 must be made of a water permeable polymer as previously described. The micro-organism 40 is too large to pass through the barrier 85 or the polymer 80 so it cannot reach the sequestered iron ion 75′ now held by the derivatized particles 75. By using the derivatized particles 75 to significantly reduce the amount of “free” iron ions 55 in the catamenial fluids captured by the sanitary napkin 150, the growth of the micro-organism 75 is eliminated or severely reduced preventing infection.

In another embodiment of the present invention, the article is a disposable diaper made in accordance with the present invention comprising low-density absorbent fibrous foam composites including a water-insoluble fiber and a superabsorbent material. The superabsorbent material has a weight amount between about 10 to 70 weight percent and the water-insoluble fiber has a weight amount between about 20 to 80 weight percent, wherein weight percent is based on total weight of the absorbent composite.

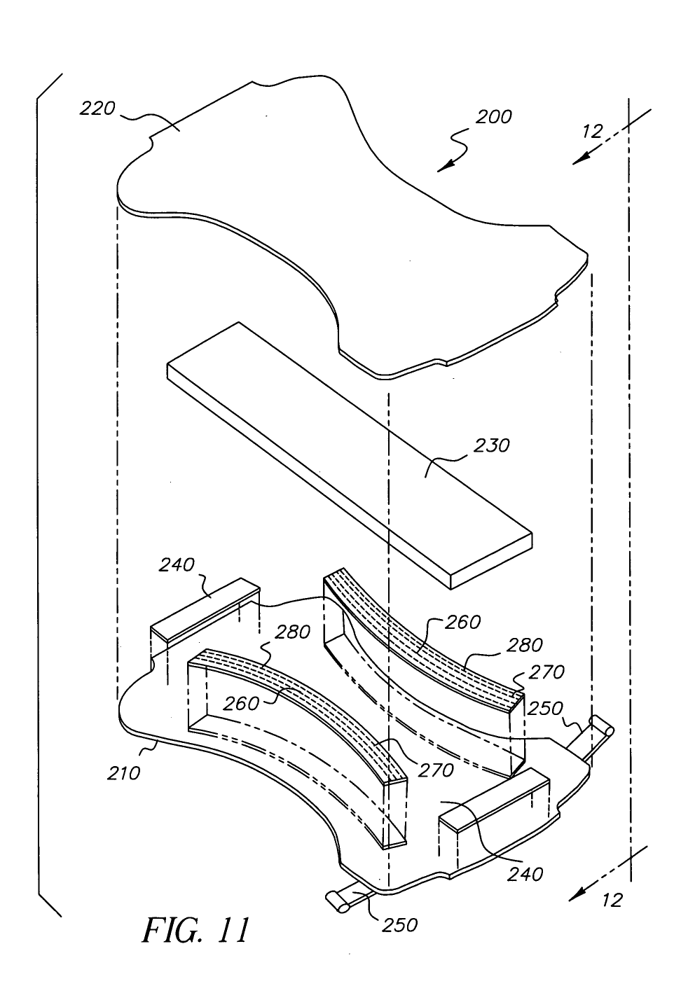

Referring to FIG. 11, disposable diaper 200 includes outer cover 210, body-side liner 220, and absorbent core 230 located between body-side liner 220 and outer cover 210. Absorbent core 230 can comprise any of the fibrous absorbent structures. Body-side liner 220 and outer cover 210 are constructed of conventional non-absorbent materials. By “non-absorbent” it is meant that these materials, excluding the pockets filled with superabsorbent, have an absorptive capacity not exceeding 5 grams of 0.9% aqueous sodium chloride solution per gram of material. Attached to outer cover 210 are waist elastics 240, fastening tapes 250 and leg elastics 260. The leg elastics 260 typically have a carrier sheet 270 and individual elastic strands 280.

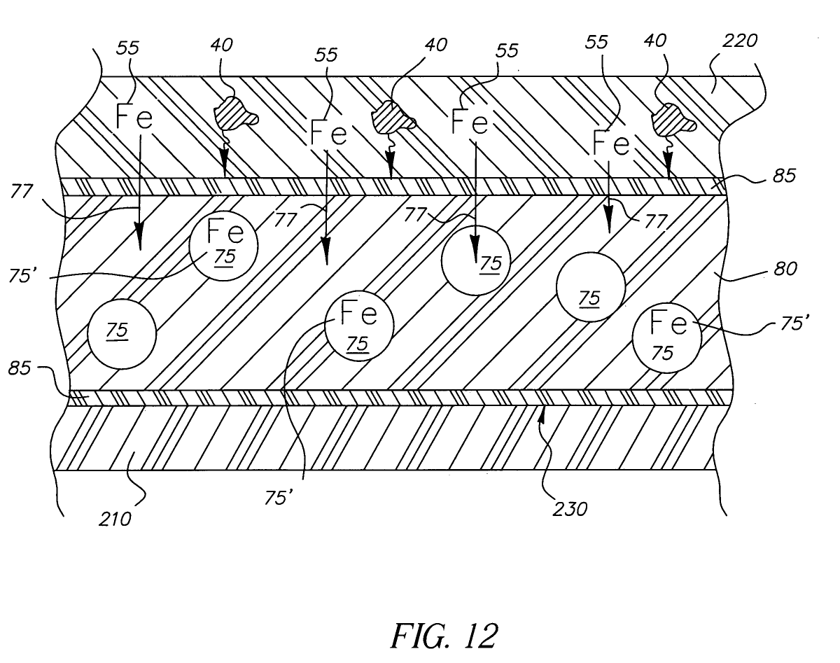

Referring to FIG. 12, there is illustrated an enlarged sectioned view of the diaper 200 shown in 11. The derivatized particles 75 are immobilized in an inner polymer 80 or the superabsorbent material disposed or incorporated in the diaper's absorbent core 230 located between body-side liner 220 and outer cover 210 and may be surrounded by a barrier layer 85. In order for the derivatized particles 75 to work properly, the inner polymer 80 containing the derivatized particles 75 must be permeable to water as previously described. A water permeable polymer permits water to move freely through the polymer 80 allowing the “free” iron ion 55 to reach and be captured by the derivatized particles 75. An additional barrier 85 may be used to prevent the micro-organism 40 from reaching the inner polymer material 80 containing the derivatized particles 75. Like the inner polymer material 80, the inner barrier layer 85 must be made of a water permeable polymer as previously described. The micro-organism 40 is too large to pass through the barrier 85 or the polymer 80 so it cannot reach the sequestered iron ion 75′ now held by the derivatized particles 75. By using the derivatized particles 75 to significantly reduce the amount of “free” iron ions 55 in the bodily fluids captured by the disposable diaper 200, the growth of the micro-organism 75 is eliminated or severely reduced preventing infection and eliminating odor.

In all the embodiments discussed above, it is preferred that the article is replaced with another identical article after the time in which the effectiveness of the article substantially decreases. The details and specifications of the articles, support structure, derivatized particles, and metal-ion sequestrant are the same as those described above for the article.

The invention has been described in detail with particular reference to certain preferred embodiments thereof, but it will be understood that variations and modifications can be effected within the scope of the invention, the present invention being defined by the claims set forth herein.

### PARTS LIST

5 bandage
10 wound
15 arm
20 support
25 pad
30 adhesive section
35 skin
40 micro-organism
45 anti-stick layer
50 arrow
55 “free” iron ion
60 arrow
65 pad
67 arrow
70 barrier layer
75 derivatized particles
75′ sequestered metal-ions
77 arrow
80 inner polymer
85 inner barrier layer
100 fibrous absorbent article
105 fibrous material
110 center core
120 tampon
130 inner layer
140 outer layer
150 sanitary napkin
160 inner layer
170 outer layer
200 disposable diaper
210 outer cover
220 body side liner
230 absorbent core
240 waste elastics
250 fastening tapes
260 leg elastics
270 carrier sheet
280 elastic strands

1. An article for inhibiting the growth of microbes in biological and physiological fluids, said article having a support structure and comprising derivatized particles having an attached metal-ion sequestrant for inhibiting the growth of said microbes, wherein the derivatized particles have a stability constant greater than 10¹⁰ with iron (III).

2. An article according to claim 1 wherein said support structure is made of fibers, fabric, textiles, plastic or paper.

3. An article according to claim 1 wherein said derivatized particles are immobilized on the support structure and have a high-affinity for biologically important metal-ions such as Mn, Zn, Cu and Fe.

4. An article according to claim 1 wherein said derivatized particles are immobilized on the support structure and have a high-selectivity for biologically important metal-ions such as Mn, Zn, Cu and Fe.

5. An article according to claim 1 wherein said derivatized particles are immobilized on the support structure and have a stability constant greater than 10²⁰ with iron (III).

6. An article according to claim 1 wherein said derivatized particles are immobilized on the support structure and have a stability constant greater than 10³⁰ with iron (III).

7. An article according to claim 1 wherein said derivatized particles comprise derivatized nanoparticles comprising inorganic nanoparticles having an attached metal-ion sequestrant, wherein said inorganic nanoparticles have an average particle size of less than 200 nm and the derivatized nanoparticles have a stability constant greater than 10¹⁰ with iron (III).

8. An article according to claim 7 wherein derivatized nanoparticles comprise inorganic nanoparticles having an attached metal-ion sequestrant, wherein said inorganic nanoparticles have an average particle size of less than 200 nm and the derivatized nanoparticles have a stability constant greater than 10²⁰ with iron (III).

9. An article according to claim 7 wherein said inorganic nanoparticles comprise silica oxides, alumina oxides, boehmites, titanium oxides, zinc oxides, tin oxides, zirconium oxides, yttrium oxides, hafnium oxides, clays, and alumina silicates.

10. An article according to claim 1 wherein said metal-ion sequestrant comprises an alpha amino carboxylate, a hydroxamate, or a catechol functional group.

11. An article according to claim 1 wherein the metal-ion sequestrant is attached to the particle, by reacting the particle with a metal alkoxide intermediate of the sequestrant having the general formula:
M(OR)₄₋ₓR′ₓ;
wherein M is silicon, titanium, aluminum, tin, or germanium;
x is an integer from 1 to 3;
R is an organic group; and
R′ is an organic group containing an alpha amino carboxylate, a hydroxamate, or a catechol.

12. An article according to claim 1 wherein said metal-ion sequestrant is attached to the particle by reacting the particle with a silicon alkoxide intermediate of the sequestrant having the general formula:
Si(OR)₄₋ₓR′ₓ;
wherein x is an integer from 1 to 3;
R is an alkyl group; and
R′ is an organic group containing an alpha amino carboxylate, a hydroxamate, or a catechol.

13. An article according to claim 1 further comprising a polymer, or polymeric layer containing said derivatized particles.

14. An article according to claim 13 wherein the polymer is permeable to water.

15. An article according to claim 13 wherein the polymer comprises one or more of polyvinyl alcohol, cellophane, water-based polyurethanes, polyester, nylon, high nitrile resins, polyethylene-polyvinyl alcohol copolymer, polystyrene, ethyl cellulose, cellulose acetate, cellulose nitrate, aqueous latexes, polyacrylic acid, polystyrene sulfonate, polyamide, polymethacrylate, polyethylene terephthalate, polystyrene, polyethylene and polypropylene or polyacrylonitrile.

16. An article according to claim 7 wherein said inorganic nanoparticles have a specific surface area of greater than 100 m²/g.

17. An article according to claim 13 further comprising a barrier layer wherein the polymeric layer is between the surface of the article and the barrier layer and wherein the barrier layer does not contain the derivatized nanoparticles.

18. An article according to claim 17 wherein the barrier layer is permeable to water.

19. An article according to claim 17 wherein the barrier layer has a thickness in the range of 0.1 microns to 10.0 microns.

20. An article according to claim 17 wherein the barrier layer comprises one or more of polyvinyl alcohol, cellophane, water-based polyurethanes, polyester, nylon, high nitrile resins, polyethylene-polyvinyl alcohol copolymer, polystyrene, ethyl cellulose, cellulose acetate, cellulose nitrate, aqueous latexes, polyacrylic acid, polystyrene sulfonate, polyamide, polymethacrylate, polyethylene terephthalate, polystyrene, polyethylene and polypropylene or polyacrylonitrile.

21. An article according to claim 17 wherein microbes cannot pass or diffuse through the barrier layer.

22. An article according to claim 1 where said article is designed to be placed against the skin of an individual.

23. An article according to claim 22 wherein said article comprises a bandage.

24. An article according to claim 23 wherein said bandage includes a liquid permeable barrier layer for allowing said biological or physiological fluids to come in contact with said derivatized particles.

25. An article according to claim 1 wherein said article comprises a diaper.

26. An article according to claim 25 wherein said diaper includes a liquid permeable membrane for allowing said nutrient to come in contact with said derivatized particles.

27. An article according to claim 1 wherein said article is designed to be placed within a living animal.

28. An article according to claim 1 wherein said article is designed to be placed within an individual.

29. An article according to claim 28 wherein said article comprises a tampon.

30. An article according to claim 28 wherein said article comprises a gauze.

31. A method for inhibiting growth of microbes in biological and physiological fluids, comprising the steps of;
a. providing an article having a support structure and derivatized particles having an attached metal-ion sequestrant for inhibiting the growth of said microbes, wherein the derivatized particles have a stability constant greater than 10¹⁰ with iron (III); and
b. placing said article in contact with said biological and/or said physiological fluid so that the growth of microbes is inhibited in said biological and/or said physiological fluid.

32. A method according to claim 31 wherein said support structure is made of fibers, fabric, textiles, plastic or paper.

33. A method according to claim 31 wherein said derivatized particles are immobilized on the support structure and have a high-affinity for biologically important metal-ions such as Mn, Zn, Cu and Fe.

34. A method according to claim 31 wherein said derivatized particles are immobilized on the support structure and have a high-selectivity for biologically important metal-ions such as Mn, Zn, Cu and Fe.

35. A method according to claim 31 wherein said derivatized particles are immobilized on the support structure and have a stability constant greater than 10²⁰ with iron (III).

36. A method according to claim 31 wherein said derivatized particles are immobilized on the support structure and have a stability constant greater than 10³⁰ with iron (III).

37. A method according to claim 31 wherein said derivatized particles comprise derivatized nanoparticles comprising inorganic nanoparticles having an attached metal-ion sequestrant, wherein said inorganic nanoparticles have an average particle size of less than 200 nm and the derivatized nanoparticles have a stability constant greater than 10¹⁰ with iron (III).

38. A method according to claim 37 wherein derivatized nanoparticles comprise inorganic nanoparticles having an attached metal-ion sequestrant, wherein said inorganic nanoparticles have an average particle size of less than 200 nm and the derivatized nanoparticles have a stability constant greater than 10²⁰ with iron (III).

39. A method according to claim 37 wherein said inorganic nanoparticles comprise silica oxides, alumina oxides, boehmites, titanium oxides, zinc oxides, tin oxides, zirconium oxides, yttrium oxides, hafnium oxides, clays, and alumina silicates.

40. A method according to claim 31 wherein said metal-ion sequestrant comprises an alpha amino carboxylate, a hydroxamate, or a catechol functional group.

41. A method according to claim 31 wherein the metal-ion sequestrant is attached to the particle, by reacting the particle with a metal alkoxide intermediate of the sequestrant having the general formula:
M(OR)₄₋ₓR′ₓ;
wherein M is silicon, titanium, aluminum, tin, or germanium;
x is an integer from 1 to 3;
R is an organic group; and
R′ is an organic group containing an alpha amino carboxylate, a hydroxamate, or a catechol.

42. A method according to claim 31 wherein said metal-ion sequestrant is attached to the particle by reacting the particle with a silicon alkoxide intermediate of the sequestrant having the general formula:
Si(OR)₄₋ₓR′ₓ;
wherein x is an integer from 1 to 3;
R is an alkyl group; and
R′ is an organic group containing an alpha amino carboxylate, a hydroxamate, or a catechol.

43. A method according to claim 31 wherein the article is replaced after a predetermined time period.

44. A method according to claim 31 wherein said support structure further comprises a polymeric layer containing said derivatized particles.

45. A method according to claim 31 where said article is designed to be placed against the skin of an individual.

46. A method according to claim 45 wherein said article comprises a bandage.

47. A method according to claim 46 wherein said bandage included a liquid permeable barrier layer for allowing said biological or physiological fluids to come in contact with said derivatized particles.

48. A method according to claim 31 wherein said article comprises a diaper.

49. A method according to claim 48 wherein said diaper includes a liquid permeable member for allowing said biological or physiological fluids to come in contact with said derivatized particles.

50. A method according to claim 31 wherein said article is designed to be placed within a living animal.

51. A method according to claim 31 wherein said article is designed to be placed within an individual.

52. A method according to claim 51 wherein said article comprises a tampon.

53. A method according to claim 51 wherein said article comprises a gauze.
